(19) United States
(12) Patent Application Publication
(10) Pub. No.: US 2016/0114127 A1
(43) Pub. Date: Apr. 28, 2016

(54) Split-Tip Catheter

(71) Applicant: Covidien LP, Mansfield, MA (US)

(72) Inventor: Sean McDermott, Hanson, MA (US)

(21) Appl. No.: 14/520,470

(22) Filed: Oct. 22, 2014

(51) Int. Cl. A61M 25/00

(52) U.S. Cl. CPC A61M 25/0074; A61M 25/0071; A61M 25/001

(57) ABSTRACT

In one aspect of the present disclosure, a catheter includes an elongate shaft, first and second tip segments, and a hydrogel. The elongate shaft has a proximal end and a distal end defining a longitudinal axis. Each of the first and second tip segments includes a proximal portion and a distal portion, the proximal portion of each of the first and second tip segments coupled to the distal end of the elongate shaft. The hydrogel is disposed along a movable portion of the first tip segment. The movable portion of the first tip segment is movable relative to the longitudinal axis under a force of volumetric expansion of the hydrogel.

### BACKGROUND

Catheters are flexible medical instruments that facilitate withdrawal and introduction of fluids from and to body cavities, ducts, and vessels. Catheters can be used, for example, in hemodialysis procedures. During some hemodialysis procedures, a multiple lumen catheter having an arterial lumen and a venous lumen is inserted into a subject's body. Blood is withdrawn through the arterial lumen of the catheter, and the removed blood is directed to a hemodialysis machine that dialyzes the blood to remove waste and toxins. The dialyzed blood is then returned to the body through a venous lumen of the catheter.

Certain hemodialysis catheters include laterally and/or longitudinally spaced arterial and venous lumen openings. Such spacing of the arterial and venous lumen openings can reduce the likelihood of blood recirculation. In these hemodialysis catheters, however, the distance and/or angle between the arterial and venous lumen openings is/are fixed. As a result, a large insertion site and/or compression of the tip of the catheter during insertion and placement is/are required, resulting in challenges associated with avoiding infection and/or accurately placing the catheter tip within a treatment site.

### SUMMARY

The present disclosure is directed to split-tip catheters having movable catheter tip segments in which the angle between the tip segments increases after placement within a body vessel for ease of insertion into the body and accurate placement of the catheter tip within a treatment site.

In one aspect of the present disclosure, a catheter includes an elongate shaft, first and second tip segments, and a hydrogel. The elongate shaft has a proximal end and a distal end defining a longitudinal axis. Each of the first and second tip segments includes a proximal portion and a distal portion, the proximal portion of each of the first and second tip segments coupled to the distal end of the elongate shaft. The hydrogel is disposed along a movable portion of the first tip segment. The movable portion of the first tip segment is movable relative to the longitudinal axis under a force of volumetric expansion of the hydrogel.

In certain embodiments, the force of volumetric expansion of the hydrogel is sufficient to separate the distal portions of the first and second tip segments from one another, in the absence of other external forces applied to the first and second tip segments, by about 0.5 cm to about 3 cm.

In some embodiments, the hydrogel is adjacent the distal end of the elongate shaft.

In certain embodiments, the movement of the movable portion of the first tip segment under the force of volumetric expansion of the hydrogel changes an included angle between the longitudinal axis and the movable portion of the first tip segment.

In certain embodiments, the hydrogel is disposed along a movable portion of the second tip segment, and the movable portion of the second tip segment is movable relative to the longitudinal axis under the force of volumetric expansion of the hydrogel.

In some embodiments, the second tip segment is fixed relative to the longitudinal axis of the elongate shaft.

The first tip segment and the elongate shaft together may define at least a portion of a first lumen and the second tip segment and the elongate shaft together may define at least a portion of a second lumen. In embodiments, the first lumen and the second lumen are fluidly isolated from one another along the elongate shaft. The first and second tip segments may each define openings that are in fluid communication with the respective first and second lumens.

In some embodiments, the hydrogel reaches its maximum volume within a period of time from about 30 seconds to about 2 hours after exposure to a water-containing fluid (e.g., a physiological fluid such as blood).

The first tip segment and the second tip segment may each have a length from about 1 cm to about 4 cm.

In some embodiments, the hydrogel is disposed between the first and second tip segments, at a junction at least partially formed by the proximal portions of the first and second tip segments.

In another aspect, a method of forming a catheter includes placing a volume of hydrogel along a movable portion of a first tip segment coupled to a distal end of an elongate shaft. The movable portion of the first tip segment is movable, under a force of volumetric expansion of the hydrogel, in a direction relative to a second tip segment coupled to the distal end of the elongate shaft.

In some embodiments, the force of volumetric expansion of the hydrogel is sufficient to separate the distal portions of the first and second tip segments from one another, in the absence of other external forces applied to the first and second tip segments, by about 0.5 cm to about 3 cm.

The method may include placing the volume of hydrogel along a movable portion of the second tip segment. The movable portion of the second tip segment is movable relative to the first tip segment under the force of volumetric expansion of the hydrogel.

In some embodiments, placing the volume of hydrogel along a movable portion of a first tip segment includes placing the volume of hydrogel at a junction at least partially formed by the first tip segment and the second tip segment.

Embodiments can include one or more of the following advantages.

In some embodiments, the hydrogel disposed along a movable portion of the first tip segment can facilitate the introduction of a split tip catheter to a treatment site without the need to mechanically maintain the first and second tip segments in a collapsed state. For example, as compared to a split tip catheter delivered to a treatment site through a sheath, the use of the hydrogel disposed along a movable portion of the first tip segment can facilitate accurate final placement of the first and second tip segments.

In certain embodiments, the first and second tip segments of the catheter have about the same cross-sectional dimension as that of the elongate shaft to facilitate the use of a small insertion site to reduce the risk of infection at the insertion site and/or for ease of maneuverability to the treatment site. Additionally or alternatively, as compared to a split tip catheter without a hydrogel, the hydrogel can control the angle of separation of the first and second tip segments to reduce the amount of movement of the first and second tip segments relative to one another at the treatment site.

Other aspects, features, and advantages will be apparent from the description, drawings, and the claims.

### BRIEF DESCRIPTION OF THE DRAWINGS

FIG. 1 is a perspective view of a catheter including movable first and second tip segments, shown in an unexpanded state.

FIG. 2 is an enlarged, perspective view of the catheter of FIG. 1, shown along the area of detail 2 identified in FIG. 1, with the movable first and second tip segments shown in an expanded state.

FIG. 3 is a perspective view of a catheter including a movable first tip segment and a fixed second tip segment, with the first and second tip segments in an unexpanded state.

FIG. 4 is an enlarged, perspective view of a portion of the catheter of FIG. 3, shown along the area of detail 4 identified in FIG. 3, with the first and second tip segments shown in an expanded state.

### DETAILED DESCRIPTION

In the following discussion, the term “proximal” refers to the portion of a structure closer to a clinician, while the term “distal” refers to the portion of the structure further from the clinician. As used herein, the term “subject” refers to a human patient or other animal. The term “clinician” refers to a doctor, nurse, or other care provider and may include support personnel. The terms “generally,” “substantially,” and “about” shall be understood as words of approximation that take into account relatively little to no variation in the modified term(s) (e.g., differing by less than ±2%).

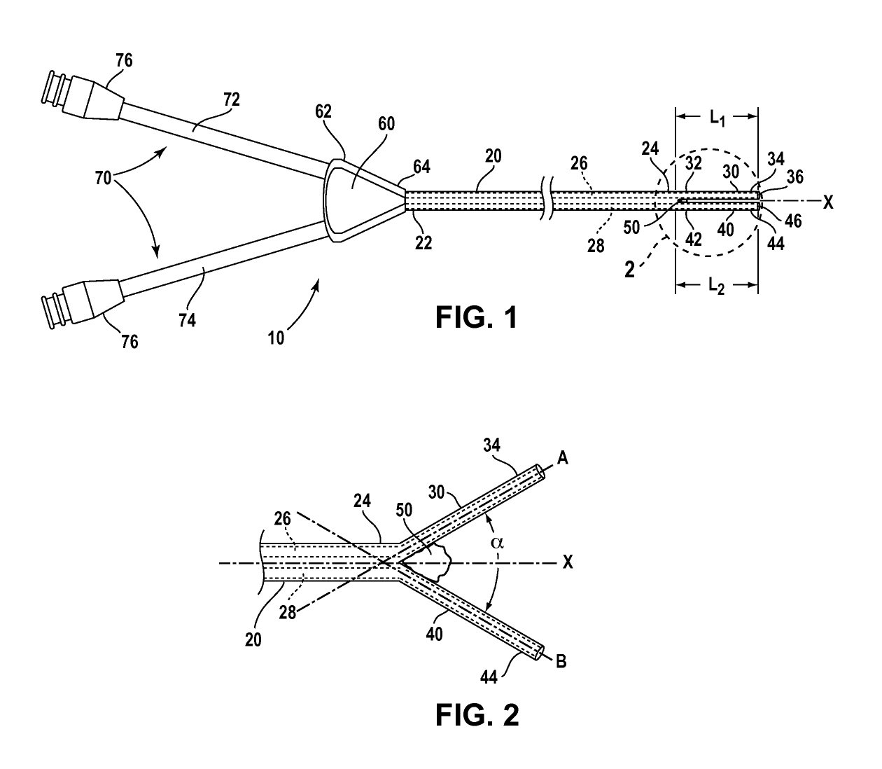

Referring now to FIGS. 1-2, a catheter 10 includes an elongate shaft 20, first and second tip segments 30, 40 distally extending from the elongate shaft 20, and a hydrogel 50 disposed between the first and second tip segments 30, 40. The catheter 10 may include a catheter hub 60 and an extension tube assembly 70 proximally extending from the elongate shaft 20. As described in further detail below, upon insertion of the first and second tip segments 30, 40 into the vasculature of a subject such that the hydrogel 50 is exposed to blood of the subject, the hydrogel 50 expands in volume to move the first and second tip segments 30, 40 from a first, substantially parallel orientation to a second, expanded orientation. As compared to the second orientation, the first orientation of the first and second tip segments 30, 40 facilitates movement of the first and second tip segments 30, 40, through the vasculature of the subject, to a desired treatment location (e.g., without the use of a sheath to hold the first and second tip segments 30, 40 in proximity to one another). As compared to the first orientation, the second, expanded orientation of the first and second tip segments 30, 40 reduces the likelihood that fluid expelled from one of the first and second tip segments 30, 40 will be recirculated into the other of the first and second tip segments. Accordingly, it should be appreciated that the hydrogel 50 disposed between the first and second tip segments 30, 40 simplifies (e.g., as compared to the use of a sheath or other movable component) delivery of the first and second tip segments 30, 40 to a treatment site while reducing the likelihood of recirculation (e.g., as compared to tip segments in close proximity to one another) of fluid between the first and second tip segments 30, 40 during treatment.

The elongate shaft 20 is a dual lumen tubular body and includes a proximal end 22 and a distal end 24 defining a longitudinal axis “X”. The elongate shaft 20 is at least partially flexible and can be at least partially formed of, for example, a biocompatible silicone and/or a thermoplastic polyurethane. For example, the elongate shaft 20 can be formed of Carbothane™, a polycarbonate-based thermoplastic polyurethane available from The Lubrizol Corporation of Wickliffe, Ohio, USA. In certain embodiments, the elongate shaft 20 is straight in the absence of an external stressor. In some embodiments, the elongate shaft 20 includes at least a pre-curved portion, in the absence of an external stressor. Such a pre-curved portion can, for example, facilitate, conforming to a body cavity or vessel into which at least a portion of the elongate shaft 20 is to be positioned.

First and second tip segments 30, 40 extend distally from the distal end 24 of the elongate shaft 20. Each of the first and second tip segments 30, 40 has a respective single lumen body. The first tip segment 30 includes a proximal portion 32 and a distal portion 34. The second tip segment includes a proximal portion 42 and a distal portion 44.

The proximal portion 32, 42 of each respective first and second tip segment 30, 40 is coupled to the distal end 24 of the elongate shaft 20. The elongate shaft 20 and the first tip segment 30 together define a first lumen 26 extending longitudinally along the catheter 10. Similarly, the elongate shaft 20 and the second tip segment 40 together define a second lumen 28 extending longitudinally along the catheter 10. The first and second lumens 26, 28 may be separate and fluidly isolated from one another such that, for example, untreated blood can be withdrawn from the subject through one of the first and second lumens 26, 28 while treated blood can be introduced into the subject through the other one of the first and second lumens 26, 28. Additionally or alternatively, each of the first and second lumens 26, 28 may have a substantially similar cross-sectional shape and/or dimension, such as a D-shape, a kidney-shape, an oblong-shape, a C-shape, a circular shape, a pie-shape, etc. The substantial similarity of the cross-sectional shape and/or dimension of the first and second lumens 26, 28 can facilitate using the catheter 10 in any orientation and/or facilitate reversing the connection of the catheter 10 to clear any obstructions (e.g., thrombogenic material) in the catheter 10.

The first and second lumens 26, 28 can extend from the proximal end 22 of the elongate shaft 20 such that the first and second lumens 26, 28 are in fluid communication with the first and second extension tubes 72, 74, respectively, and through the distal portions 34, 44 of the first and second tip segments 30, 40 such that the first and second lumens 26, 28 are in fluid communication with a body cavity or vessel in which the first and second tip segments 34, 40 are positioned. Distal portions 34, 44 of the first and second tip segments 30, 40 define openings 36, 46 in fluid communication with a respective one of the first and second lumens 26, 28 such that the first and second lumens 26, 28 are in fluid communication with a body cavity or vessel in which the first and second tip segments 34, 40 are positioned.

The length “L₁” of the first tip segment 30 may be substantially equal to the length “L₂” of the second tip segment 40, and the distal portions 34, 44 of the first and second tip segments 30, 40 may be coterminous with each other. For example, the length “L₁” can be substantially equal to the length “L₂” such that the first tip segment 30 and the second tip segment 40 can be interchangeable with one another to facilitate flow reversal through the catheter 10. At least a portion of the length “L₁,” L₂” of each of the first and second tip segments 30, 40 defines a movable portion of the first and second tip segments 30, 40. In some embodiments, the length “L₁,” L₂” of each of the first and second tip segments 30, 40 is from about 1 cm to about 4 cm to facilitate, for example, adequate lateral separation of the distal portions 34, 44 of the respective first and second tip segments 30, 40 upon expansion of the hydrogel 50.

The hydrogel 50 is disposed along the movable portion of the first tip segment 30 and the movable portion of the second tip segment 40. For example, the hydrogel 50 can be disposed at a junction at least partially formed by the proximal portions 32, 42 of the respective first and second tip segments 30, 40, adjacent the distal end 24 of the elongate shaft 20. Expansion of the hydrogel 50 (e.g., upon exposure to moisture such as a physiological fluid) moves the movable portion of the first and second tip segments 30, 40 to change an included angle “α” between the longitudinal axis “X” of the elongate shaft 20 and the movable portions of the first and second tip segments 30, 40. In the angled configuration, longitudinal axes “A,” “B” defined by the first and second tip segments 30, 40 are disposed at respective angles with respect to the longitudinal axis “X” of the elongate shaft 20 to define the included angle “α” therebetween. The amount of volumetric expansion and the position of the hydrogel 50 between the first and second tip segments 30, 40 impacts the size of the included angle “α” between the first and second lumens 26, 28 defined within the first and second tip segments 30, 40.

In some embodiments, expansion of the hydrogel 50 moves the first and second tip segments 30, 40 relative to one another such that the distal portions 34, 44 of the respective first and second tip segments 30, 40 are laterally spaced relative to one another by about 0.5 cm to about 3 cm in the absence of external forces applied to the first and second tip segments 30, 40. Such lateral spacing of the distal portions 34, 44 of the respective first and second tip segments 30, 40 can reduce the likelihood of recirculation of fluid exiting one of the first and second tip segments 30, 40 into the other of the first and second tip segments 30, 40 during, for example, hemodialysis treatment.

The hydrogel 50 is formed from a biocompatible material that volumetrically expands under physiological conditions. For example, the hydrogel 50 can include one or more of the following materials: gelatin material, polyethylene glycol, and sodium alginate. In some embodiments, the hydrogel is 100 percent polyethylene glycol diacrylate (PEGDA) with a polymer-to-weight percent of 20 with a stiffness of about 350 kPa and, upon exposure to physiological conditions, is expandable to about 10 times its initial volume.

In certain embodiments, the contact area between the hydrogel 50 and the first and second tip segments 30, 40, in the unexpanded state of the hydrogel 50, is between about 30 mm² to about 50 mm². It should be appreciated that this range of contact area between the hydrogel 50 and the first and second tip segments 30, 40 can exert sufficient force on the first and second tip segments 30, 40 upon expansion of the hydrogel 50 to achieve sufficient separation of the first and second tip segments 30, 40 at the treatment site. Additionally or alternatively, it should be appreciated that this range of contact area between the hydrogel 50 and the first and second tip segments 30, 40 can be achieved using a volume of the hydrogel 50 does not produce substantial separation of the first and second tip segments 30, 40 when the hydrogel 50 is in the unexpanded state, thus facilitating delivery of the first and second tip segments 30, 40 to the treatment site.

The hydrogel 50 may reach its maximum volume within a period of time from about 30 seconds to about 2 hours after exposure to a physiological fluid. This range of expansion time is typically less than the time between initial exposure of the first and second tip segments 30, 40 to physiological fluid (e.g., upon initial insertion into the vasculature of the subject) and placement of the first and second tip segments 30, 40 at the treatment site. Thus, for example, expansion of the hydrogel 50 to its maximum volume within a period of time from about 30 seconds to about 2 hours after exposure to a physiological fluid can facilitate movement of the first and second tip segments 30, 40 to the treatment site without significant separation of the first and second tip segments 30, 40 as the first and second tip segments 30, 40 are maneuvered to the treatment site.

The catheter hub 60 is dimensioned for manual engagement by a clinician. The catheter hub 60 includes a proximal housing section 62 adjacent first and second extension tubes 72, 74 of the extension tube assembly 70, and a distal housing section 64 adjacent the elongate shaft 20. The proximal housing section 62 is attachable to the first and second extension tubes 72, 74, and the distal housing section 64 is attachable to the elongate shaft 20 in secured relation therewith.

Each of the first and second extension tubes 72, 74 may include a respective luer adapter 76 at its free end for connection to inflow and outflow ports of a treatment device. Additionally or alternatively, a clamp may be mounted about each of the first and second extension tubes 72, 74 to control fluid flow.

In an exemplary method of use, the catheter 10, with the hydrogel 50 in the unexpanded state, is inserted into a target vessel, for example, by advancing the elongate shaft 20 along a guidewire until the first and second tip segments 30, 40 are adjacent or within the treatment site. After a predetermined time of exposure to the subject's bodily fluids, the hydrogel 50 expands, and the movable portions of the first and second tip segments 30, 40 are moved relative to the longitudinal axis “X” of the elongate shaft 20 under the force of the volumetric expansion of the hydrogel 50. Movement of the movable portions of the first and second tip segments 30, 40 changes the included angle “α” defined between the first and second tip segments 30, 40 relative to the longitudinal axis “X” of the elongate shaft 20, separating the distal portions 34, 44 of the first and second tip segments 30, 40 from one another.

The first and second extension tubes 72, 74 are coupled to inflow and outflow ports of an extracorporeal treatment device (e.g., a hemodialysis machine), and the treatment device is activated. Blood is drawn from the vasculature through one of the first and second tip segments 30, 40 and directed through a respective one of the first and second lumens 26, 28 to the treatment device. Treated blood is returned from the treatment device, through the other of the first and second lumens 30, 40, and delivered from a respective one of the first and second tip segments 30, 40 into the vasculature of the subject.

In some embodiments, a method of forming the catheter 10 includes splitting the elongate shaft 20 along a septum separating first and second lumens such that first and second tip segments 30, 40 extend distally from the elongate shaft 20. A volume of the hydrogel 50 can be placed between the first and second tip segments 30, 40, along a movable portion of the first tip segment 30 and/or a movable portion of the second tip segment 40 such that the movable portions of the first and second tip segments 30, 40 are movable, under a force of volumetric expansion of the hydrogel 50, as described above.

In certain embodiments, a method of forming the catheter 10 includes injection molding the first and second tip segments 30, 40 and bonding (e.g., heat bonding) the first and second tip segments 30, 40 to the elongate shaft 20. A volume of the hydrogel 50 can be placed between the first and second tip segments 30, 40, along a movable portion of the first tip segment 30 and/or a movable portion of the second tip segment 40 such that the movable portions of the first and second tip segments 30, 40 are movable, under a force of volumetric expansion of the hydrogel, as described above.

While certain embodiments have been described, other embodiments are possible.

For example, while catheters have been shown as having openings at the distal-most end of the distal portion of first and second tip segments, other configurations are additionally or alternatively possible. For example, catheters can additionally or alternatively include first and second tip segments defining openings disposed about a side wall of the distal portion of each of the first and second tip segments, and/or the distal portions of the first and second tip segments may define multiple openings.

As another example, while catheters have been described as including hydrogels at junction formed by proximal portions of first and second tip segments, in contact with each of the first and second tip segments, other configurations are additionally or alternatively possible. For example, the hydrogel may be disposed on only one of the first and second tip segments, and/or may be distally spaced from the junction of the proximal portions of the first and second tip segments and the distal end of the elongate shaft.

As yet another example, while catheters have been described as having first and second tip segments that each have movable portions, other configurations are additionally or alternatively possible. For example, one of the first and second tip segments may be fixed relative to the longitudinal axis of the elongate shaft.

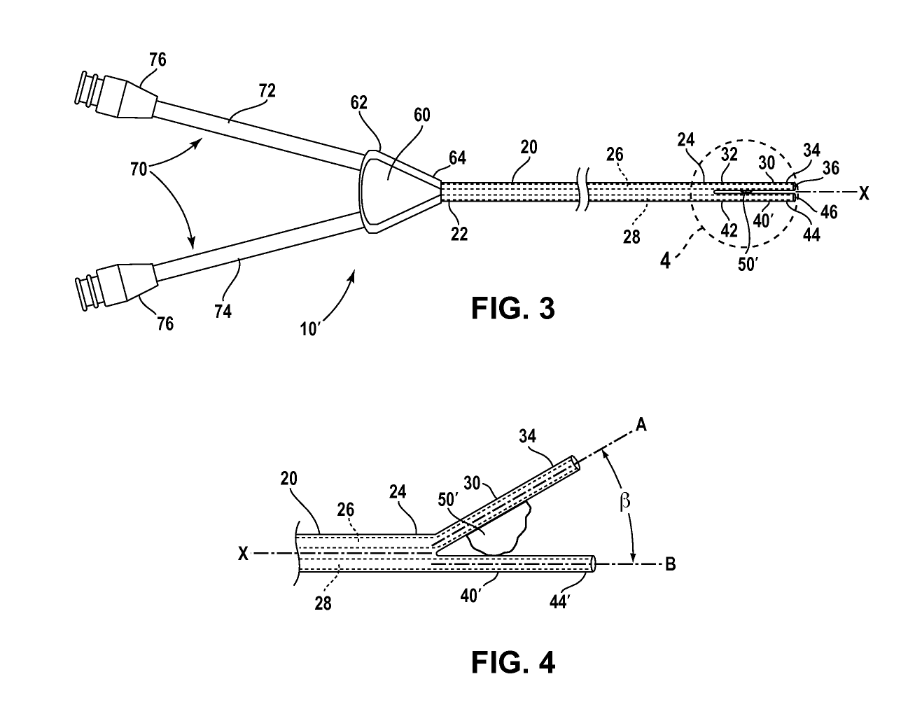

With reference to FIG. 3, a catheter 10′ includes an elongate shaft 20, first and second tip segments 30, 40′ extending distally from the elongate shaft 20, a hydrogel 50′ disposed between the first and second tip segments 30, 40′, and a catheter hub 60 and an extension tube assembly 70 extending proximally from the elongate shaft 20. Except as provided for below, the primed element numbers are similar to the respective unprimed element numbers described above with respect to FIGS. 1-2. For example, the hydrogel 50′ is similar to hydrogel 50, except as provided for below.

The hydrogel 50′ is disposed, in an unexpanded state, along the movable portion of the first tip segment 30 and is distally spaced from the distal end 24 of the elongate shaft 20. The second tip segment 40′ is fixed relative to the longitudinal axis “X” of the elongate shaft 20 such that the second tip segment 40′ remains in the same position relative to the longitudinal axis “X” upon expansion of the hydrogel 50′. In some embodiments, the second tip segment 40′ is substantially parallel to the longitudinal axis “X” of the elongate shaft 20.

As shown in FIG. 4, the first tip segment 30 is movable to an angled configuration, after the hydrogel 50′ has been exposed to a physiological fluid and the force of volumetric expansion of the hydrogel 50′ has moved the movable portion of the first tip segment 30 relative to the longitudinal axis “X” of the elongate shaft 20. The force of volumetric expansion of the hydrogel 50′ is sufficient to separate the distal portions 34, 44′ of the first and second tip segments 30, 40′ from one another. The movement of the movable portion of the first tip segment 30 changes an included angle “β” between the longitudinal axis “X” of the elongate shaft 20 and the movable portion of the first tip segment 30. In the angled configuration, longitudinal axis “A” defined by the first tip segment 30 is disposed at an angle with respect to the longitudinal axis “X” of the elongate shaft 20 and longitudinal axis “B” defined by the second tip segment 40′, which has remained substantially stationary during expansion of the hydrogel 50′, to define the included angle “β” therebetween.

A number of embodiments have been described. Nevertheless, it will be understood that various modifications may be made without departing from the spirit and scope of the disclosure. Accordingly, other embodiments are within the scope of the following claims.

What is claimed is:

1. A catheter comprising:
an elongate shaft having a proximal end and a distal end, the proximal end and the distal end defining a longitudinal axis;
first and second tip segments, each tip segment including a proximal portion and a distal portion, the proximal portion of each tip segment coupled to the distal end of the elongate shaft; and
a hydrogel disposed along a movable portion of the first tip segment, the movable portion of the first tip segment movable relative to the longitudinal axis under a force of volumetric expansion of the hydrogel.

2. The catheter of claim 1, wherein the force of volumetric expansion of the hydrogel is sufficient to separate the distal portions of the first and second tip segments from one another, in the absence of other external forces applied to the first and second tip segments, by about 0.5 cm to about 3 cm.

3. The catheter of claim 1, wherein the hydrogel is adjacent the distal end of the elongate shaft.

4. The catheter of claim 1, wherein movement of the movable portion under the force of volumetric expansion of the hydrogel changes an included angle between the longitudinal axis and the movable portion of the first tip segment.

5. The catheter of claim 1, wherein the hydrogel is disposed along a movable portion of the second tip segment, the movable portion of the second tip segment movable relative to the longitudinal axis under the force of volumetric expansion of the hydrogel.

6. The catheter of claim 1, wherein the second tip segment is fixed relative to the longitudinal axis of the elongate shaft.

7. The catheter of claim 1, wherein the first tip segment and the elongate shaft together define at least a portion of a first lumen and the second tip segment and the elongate shaft together define at least a portion of a second lumen.

8. The catheter of claim 7, wherein the first lumen and the second lumen are fluidly isolated from one another along the elongate shaft.

9. The catheter of claim 7, wherein the first and second tip segments each define openings, the openings of the first and second tip segments in fluid communication with the respective first and second lumens.

10. The catheter of claim 1, wherein the hydrogel reaches its maximum volume within a period of time from about 30 seconds to about 2 hours of exposure to a water-containing fluid.

11. The catheter of claim 1, wherein the first tip segment and the second tip segment each has a length from about 1 cm to about 4 cm.

12. The catheter of claim 1, wherein the hydrogel is disposed between the first and second tip segments, at a junction at least partially formed by the proximal portions of the first and second tip segments.

13. A method of forming a catheter, the method comprising:
placing a volume of hydrogel along a movable portion of a first tip segment coupled to a distal end of an elongate shaft, the movable portion of the first tip segment movable, under a force of volumetric expansion of the hydrogel, in a direction relative to a second tip segment coupled to the distal end of the elongate shaft.

14. The method of claim 13, wherein the force of volumetric expansion of the hydrogel is sufficient to separate the distal portions of the first and second tip segments from one another, in the absence of other external forces applied to the first and second tip segments, by about 0.5 cm to about 3 cm.

15. The method of claim 13, further comprising placing the volume of hydrogel along a movable portion of the second tip segment, the movable portion of the second tip segment movable relative to the first tip segment under the force of volumetric expansion of the hydrogel.

16. The method of claim 13, wherein placing the volume of hydrogel along a movable portion of a first tip segment includes placing the volume of hydrogel at a junction at least partially formed by the first tip segment and the second tip segment.
